(19) United States
(12) Patent Application Publication
(10) Pub. No.: US 2025/0275700 A1
(43) Pub. Date: Sep. 4, 2025

(54) METHOD AND SYSTEM FOR MONITORING COGNITIVE STATE OF AN INDIVIDUAL

(71) Applicant: ZE CORRACTIONS LTD., Mevasseret Zion (IL)

(72) Inventors: Eldad Izhak HOCHMAN, Raanana (IL); Zohar REHNICHER, Jerusalem (IL)

(21) Appl. No.: 18/863,701

(22) PCT Filed: Jun. 14, 2023
(86) PCT No.: PCT/IL2023/050610

§ 371 Date: Nov. 7, 2024

Related U.S. Application Data
(60) Provisional application No. 63/352,306, filed on Jun. 15, 2022.

(51) Int. Cl. A61B 5/16; A61B 5/00; A61B 5/05; A61B 5/11; A61B 5/18

(52) U.S. Cl. CPC A61B 5/165; A61B 5/0002; A61B 5/05; A61B 5/1114; A61B 5/18; A61B 5/4842; A61B 5/4845; A61B 2503/22

(57) ABSTRACT

A system is presented for monitoring a cognitive state of an individual during a certain activity of the individual. The system comprises a control system in data communication with measured data provider to receive therefrom measured data indicative of motion patterns originated in at least one body part of the individual and being detected over time by a sensing system operable with predetermined measuring conditions. The control system is responsive to said measured data to apply model-based processing to the measured data and determine a cognitive state of the individual, said model-based processing comprising applying to said measured data a number of predetermined models, each of the predetermined models describing a relation between motion patterns, originated in at least one body part of an individual and being collected under predetermined measuring conditions, and an associated characteristic factor of a cognitive state of the individual.

### TECHNOLOGICAL FIELD AND BACKGROUND

The present disclosure is in the field of monitoring techniques and relates to a method and system for monitoring cognitive state of an individual.

Various techniques have been developed aimed at monitoring individual's state/condition based on brain commands' detection, using EEG signals and/or movements. For example, U.S. Pat. Nos. 10,413,246 and 11,141,113, describe earlier techniques of the co-inventor of the present application, dealing with controlling machine operation via monitoring cognitive brain commands of a user, in order to detect the motor command related data corresponding to a condition of user's detection of error in his/her cognitive brain command.

### GENERAL DESCRIPTION

There is a need in the art for a novel approach in determining an individual's cognitive state during a certain activity of the individual, which can be identified from motion patterns characterizing various movements performed by part(s) of the individual's body during said certain activity.

Typically, the individual's activity defines a “normal” cognitive condition/state in association with/assigned to said activity. For example, movements performed by an individual include goal directed movements related to the individual's activity. These movements may be conscious or unconscious and performed in order to achieve a certain goal (e.g. grasping an object) or may be associated with an immediate result of trying to achieve a certain goal (muscle movement in response to imminent danger). Typically, such goal directed movement is not a side effect of a particular physical condition/factor (e.g. tremor in Parkinson's patients or falling of the head and closing eyelids when tired).

When a movement is planned or is being executed, events in the individual's brain or events in the individual's environment require constant updating of the movement plan. The brain has performance control systems of the brain that detect the need to adapt action plans. These performance control systems are influenced by factors that affect an individual's emotional or cognitive condition/state.

However, because of the nature of the brain signals that reflect operation of the performance control systems, direct recording the operational condition of the brain in the performance control systems does not allow to know the reason/source for the change in the emotional or cognitive condition.

Various techniques have been described in the scientific literature, according to which fluctuations in the cognitive or emotional condition (e.g. in ability to take correct decisions and produce accurate movements or control one's emotions in a timely manner) can be identified using electrophysiological methods (EEG). These methods, however, cannot discern the cause for such fluctuations.

In fact, when one wants to identify what is the cause of the emotional or cognitive condition alteration of an individual, one is forced to use different measurement techniques to evaluate each specific factor as an option to be a source of said alterations of the cognitive condition. For example, cameras in vehicles are suggested to be used to monitor driver's head position and eyelid closure, as a means of measuring fatigue and pupil movements indicative of driver's inattention. Breath analyzers can be used to measure the level of alcohol in the driver's blood.

The inventors have found that an individual's cognitive state during certain activity, i.e. being a source for a change in the emotional or cognitive condition of the individual from a normal condition for a given activity, can be extracted from motions originated in at least one body part of the individual. In this connection, it should be understood, that the technique of the present disclosure provides for identifying the factor characterizing the individual's cognitive state (from a variety of different factors said that may affect changes in the individual's cognitive condition). Such factor is a measure/characteristic of the individual's cognitive state.

The technique utilizing analysis of various motion patterns originated at the individual's body in association with individual's cognitive condition has been developed by the inventors of the present application and described in PCT/IL2021/051464 assigned to the assignee of the present application. This earlier technique of the inventors deals with cognitive error detection, i.e. extracting from motion patterns characterizing a certain activity of the individual and a cognitive error detection by said individual in said activity.

The present disclosure is aimed at direct determination of the individual's cognitive state of interest, which is important while monitoring various activities of individuals, via detection and analysis of motion patterns originated at part(s)/portion(s) of individual's body.

It should be noted that for the purposes of the present application, the term “cognitive state” used herein refers to individual's brain state uniquely characterizing a cause/reason of a change/alteration of his/her cognitive condition from a normal condition associated with/related to the individual's current activity. For example, such activity as watching a screen is associated/described by a respective “normal” cognitive condition. A change in the cognitive condition from the normal one may be associated with many factors, such as drug use, acute physical condition, exhaustion, intoxication, fatigue, stress, attention withdrawal, motion sickness etc., each factor characterizing a different cognitive state.

It should be understood that for the purposes of the present disclosure the motion patterns being sensed and analyzed may or may not relate to any goal directed movement, as well as the extracted/derived factor may or may not be related to individual's brain detection of any error.

It should also be noted that the term “certain activity” of an individual is not limited to a physical active condition (i.e. execution of any physical action involving movements performed by an individual), and this term should thus be interpreted broadly covering also so-called “physically passive” condition, i.e. resting, sitting, sleeping.

In order to identify the specific cognitive state (e.g. fatigue), feature(s) uniquely associated with the respective factor are to be identified in the measured data describing the cognitive condition of the individual during certain activity. This measured data is formed by motion patterns originated in at least one body part of the individual and collected over time from at least one sensor unit.

It should be noted that the meaning of the term “sensor unit” should not be limited to “motion sensor”. Indeed, there is a variety of sensors which are not motion sensors per se and are capable of measuring one or more parameters from which data indicative of motion can be derived. Such sensors include for example a piezoelectric sensor, a pressure sensor, a force sensor. Hence, for the purposes of the present application, sensor unit may be of any known suitable type providing sensing data which can be transformed into motion data. It should also be understood that measurements of the motion data/pattern originated at the body part of the individual may be implemented by sensor unit (not necessarily being “motion sensor”) physically attached to the body part or sensor unit (being motion sensor or not) measuring motion data/pattern of a device/element interacting with the body part of the individual and being driven by said body part.

Thus, the present disclosure provides a novel technique for monitoring individual's cognitive condition during certain activity to identify a cognitive state of the individual characterized by a corresponding factor affecting a change in movement control performed by individual's brain. The inventors have found that the same sensing data indicative of motion patterns originated at a certain body part of the individual can be used to identify and distinguish between different factors describing/relating to different cognitive states of individual (e.g., intoxication, fatigue) during the individual's activity.

The technique of the present disclosure includes monitoring operation of the brain performance of an individual during a certain activity of the individual via detection and analysis of sensing data indicative of motion pattern(s) originated in at least one body part of the individual to identify a cognitive state of the individual, being a cause/source of a change of the cognitive condition from a normal condition assigned to said activity of the individual.

According to the technique of the present disclosure, motion patterns detected over time on at least one body part of an individual (so-called “body movement”) are processed and analyzed to identify relevant features within the movement. Considering for example monitoring the cognitive state of an individual while driving a vehicle (e.g., car), the motion patterns that can be measured and analyzed may include those associated with the angle of a steering wheel and/or image data indicative of images/photographs acquired from the individual's body by a camera and/or motion patterns detected by a radar that transmits signals to the body and being indicative of movements in the vehicle.

The analysis of the measured data (motion patterns) is implemented in the present disclosure by utilizing a predetermined number of models (machine learning models) each being trained to describe a relation between motion patterns, originated in at least one body part of individual and being collected under predetermined measuring conditions, and an associated characteristic factor of a cognitive state of the individual.

Thus, according to one broad aspect of the present disclosure, it provides a system for monitoring a cognitive state of an individual during a certain activity of the individual, the system comprising:

a control system configured for data communication with a measured data provider to receive therefrom measured data indicative of motion patterns originated in at least one body part of the individual and being detected over time by a sensing system operable with predetermined measuring conditions, the control system being configured and operable to process the measured data by applying thereto model-based processing and determine a cognitive state of the individual, said model-based processing comprising applying to said measured data a number of predetermined models, each of the predetermined models describing a relation between motion patterns, originated in at least one body part of an individual and being collected under predetermined measuring conditions, and an associated characteristic factor of a cognitive state of the individual.

The models used include machine learning models obtained in training and learning procedures.

The at least one predetermined factor being monitored may correspond to the at least one of the following cognitive states: drunkenness, fatigue, exhaustion, stress, motion sickness, inattention, development of acute physical condition affecting cognitive state.

The control system is typically a computer system comprising data input and output utilities, memory, and a processor and analyzer. The processor and analyzer is adapted to be responsive to the measured data and to analyze said measured data by applying thereto at least one selected model from said predetermined number of models, in accordance with the cognitive state being monitored, and determine the cognitive state by identifying in the measured data at least one feature uniquely associated with the respective characteristic factor describing a cognitive condition of the individual during certain activity.

In some embodiments, the processor and analyzer is configured to analyze said measured data and, upon identifying a predetermined change in a cognitive condition of the individual being monitored, applying said at least one selected model to said measured data to determine the characteristic factor affecting said change of the cognitive condition, and extracting the corresponding cognitive state of said individual. For example, the processor and analyzer may be configured and operable to quantify a degree of change of the cognitive condition of the individual associated with said corresponding cognitive state, and selectively perform said applying of the predetermined model to the measured data, upon identifying that said degree of change is above predetermined level.

In some other embodiments, the control system is configured and operable to be responsive to the measured data indicative of the motion patterns corresponding to the predetermined level/degree of change of the cognitive condition to initiate the model-based processing to the measured data using the at least one predetermined model.

In some embodiments, the control system is configured and operable for data communication with a storage device storing said number of predetermined models via a communication network.

In some embodiments, the monitoring system also includes a sensing system comprising a number N of sensor units (N≥1), each sensing unit comprising one or more sensors and being configured and operable with predetermined measuring conditions to detect data indicative of motion patterns originated in at least one body part of the individual and generate said measured data indicative of the motion patterns being detected.

As described above, the characteristic factor characterizing the cognitive state may comprise at least one of the following: drug use, inattention, acute physical condition, exhaustion, intoxication, fatigue, stress, attention withdrawal, motion sickness, drunkenness.

Considering the example of monitoring the cognitive state of an individual while driving a vehicle, the measured data may be indicative of the motion pattern detected from measurements of steering wheel angular velocity. The characteristic factor to be identified may be fatigue or drunkenness. The at least one feature uniquely associated with the characteristic factor may comprise at least one of the following: longest move above mean determined as a length of the longest sub-signal in the motion pattern that is higher than an average value of a measured motion pattern signal; maximum frequency of a measured motion pattern signal; tailedness of frequency distribution of a motion pattern signal being measured; and a ratio of values in the motion pattern signal which are higher than 2.5 times standard deviation of the signal.

According to another broad aspect of the invention, it provides a method for monitoring a cognitive state of an individual during a certain activity of the individual, the method comprising:
providing measured data indicative of motion patterns originated in at least one body part of the individual and being detected over time under predetermined measuring conditions,
processing the measured data by applying thereto model-based processing and determining a cognitive state of the individual, said model-based processing comprising applying to said measured data a number of predetermined models, each of the predetermined models describing a relation between motion patterns, originated in at least one body part of an individual and being collected under predetermined measuring conditions, and an associated characteristic factor of a cognitive state of the individual.

According to yet another broad aspect of the present disclosure, it provides a system for use in monitoring a cognitive state of an individual during a certain activity of the individual, the system being a computer system comprising:
modeling utility configured and operable to apply machine learning processing to sensing data and generate at least one model interpreting said sensing data in association with at least one predetermined characteristic factor corresponding to a cognitive state of an individual, wherein:
said sensing data corresponds to measurements performed by N (N≥1) sensing units in association with body parts of individuals, each measurement being indicative of motion patterns originated at a respective body part of an individual, said sensing data comprising a train data formed by at least first and second sets of motion patterns measured on, respectively, at least one first group of a plurality of M individuals whose cognitive state is characterized by said at least one predetermined characteristic factor, and at least one second group of a plurality of G individuals whose cognitive state is free of said at least one predetermined characteristic factor;
said modelling utility is configured and operable to receive said train data and apply model learning and training processing thereto and determine, for each of said at least one predetermined characteristic factor, a set of L characteristics (UC₁ . . . . UC_{L}) uniquely describing a change in a cognitive condition of an individual corresponding to a certain cognitive state of said individual.

As indicated above, the at least one predetermined characteristic factor being monitored may correspond to the at least one of the following cognitive states: drunkenness, fatigue, exhaustion, stress, motion sickness, inattention, development of acute physical condition affecting cognitive state.

In some embodiments, the first and second sets of motion patterns correspond to measurements performed on the at least one first group of the individuals whose cognitive state is characterized by said at least one characteristic factor, and the at least one second group of the individuals whose cognitive state is characterized by a second abnormality factor to be monitored and distinguished from said at least one characteristic factor.

The model learning and training processing comprises classification of said first and second sets of motion patterns to identify first and second sets of L characteristics (UC₁ . . . . UC_{L})₁ and (UC₁ . . . . UC_{L})₂ uniquely distinguishing the motion patterns measured on the individuals of the two groups.

The distinguishing characteristic may be determined by one of the following: (i) absence and presence of a certain motion pattern relating feature in the first and second sets, respectively; or (ii) presence of a certain motion pattern relating feature in both the first and second sets but at different levels (values) according to a predefined criteria.

In some embodiments, the first and second sets of motion patterns correspond to measurements performed on the at least one first group of the individuals whose cognitive state is characterized by said at least one predetermined characteristic factor, and the at least one second group of the individuals being a control group whose cognitive state is classified as a normal state.

### BRIEF DESCRIPTION OF THE DRAWINGS

In order to better understand the subject matter that is disclosed herein and to exemplify how it may be carried out in practice, embodiments will now be described, by way of non-limiting examples only, with reference to the accompanying drawings, in which:

FIGS. 1A to 1C exemplify the principles of the technique of the present disclosure, wherein FIG. 1A is a block diagram of an exemplary model creation system; FIG. 1B is a block diagram of an exemplary monitoring system using the so-created model for determination of a cognitive state of an individual, and FIG. 1C exemplifies more specifically the database system configuration (i.e. library with its interpretation engine);

FIGS. 2A to 2C show flow diagrams exemplifying the technique of the present disclosure, wherein FIG. 2A exemplifies the method for identifying the factor affecting a change of the cognitive condition and evaluating/quantifying the level of such factor; and FIGS. 2B and 2C show two examples of the model creation (training/learning) enabling taxonomy of different levels of the effect of the same factor on the change in the cognition condition of an individual;

FIGS. 3A-3B, 4A-4B, 5A-5B and 6A-6B show experimental data demonstrating comparison of two cognitive states of fatigue and drunkenness, using different unique characteristics of motion patterns assigned to the different cognitive states, wherein

FIGS. 3A, 4A, 5A and 6A show distribution of the value of the respective unique characteristic for the different cognitive states, and FIGS. 3B, 4B, 5B and 6B show exemplary signals with different value of said unique characteristic.

### DETAILED DESCRIPTION OF EMBODIMENTS

In the following detailed description, numerous specific details are set forth in order to provide a thorough understanding of the invention. While specific details are provided to enable a thorough understanding of the invention, those skilled in the art will appreciate that the invention may be practiced without these details. Additionally, well-known methods, procedures, and components are not described in detail to avoid obscuring the invention. Finally, any reference to a method, system, or non-transitory computer readable medium should be interpreted as including related aspects of the invention.

The terms “computer”, “processor”, and “controller” should be expansively construed to cover any kind of electronic device with data processing capabilities, including, by way of non-limiting example, a personal computer, a server, a computing system, a communication device, a processor (e.g. digital signal processor, DSP), a microcontroller, a field programmable gate array (FPGA), cloud computing server, an application specific integrated circuit (ASIC), a smartphone, an electronic control unit (ECU) of a vehicle, an and so on. Unless stated otherwise, the terms “computer”, “processor”, and “controller” may also include a combination of several modules (e.g., several central processing units, CPUs), which operate together toward a goal. Unless specifically stated otherwise, as apparent from the following discussions, it is appreciated that throughout the specification discussions utilizing terms such as “processing”, “calculating”, “computing”, “determining”, “generating”, “setting”, “configuring”, “selecting”, “defining”, or the like, include actions and/or processes of a computer that manipulate and/or transform data into other data. That data is represented as physical quantities, e.g., such as electronic or electromagnetic quantities, and/or said data representing physical objects.

It is appreciated that certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination. In embodiments of the presently disclosed subject matter one or more steps illustrated in the figures may be executed in a different order and/or one or more groups of steps may be executed simultaneously. The figures illustrate a general schematic of the system architecture in accordance with an embodiment of the presently disclosed subject matter. Each module in the figures can be made up of any combination of software, hardware and/or firmware that performs the functions as defined and explained herein. The modules in the figures may be centralized in one location or dispersed over more than one location.

Any reference in the specification to a method should be applied mutatis mutandis to a system capable of executing the method. Any reference in the specification to a method which can be executed by a computer should be applied mutatis mutandis to a non-transitory computer readable medium that stores instructions that once executed by a computer result in the execution of the method. All the details, variations, optional features, optional steps which are discussed with respect to a system are also applicable, mutatis mutandis, to such a corresponding method (and non-transitory computer readable medium, where applicable), and vice versa.

The present disclosure provides a data analysis technique for analyzing various motion patterns originated at one or more body parts of an individual to identify factor(s) that affect(s) a change in the individual's cognitive condition and characterize the individual's cognitive state associated with/caused by characteristic factor(s).

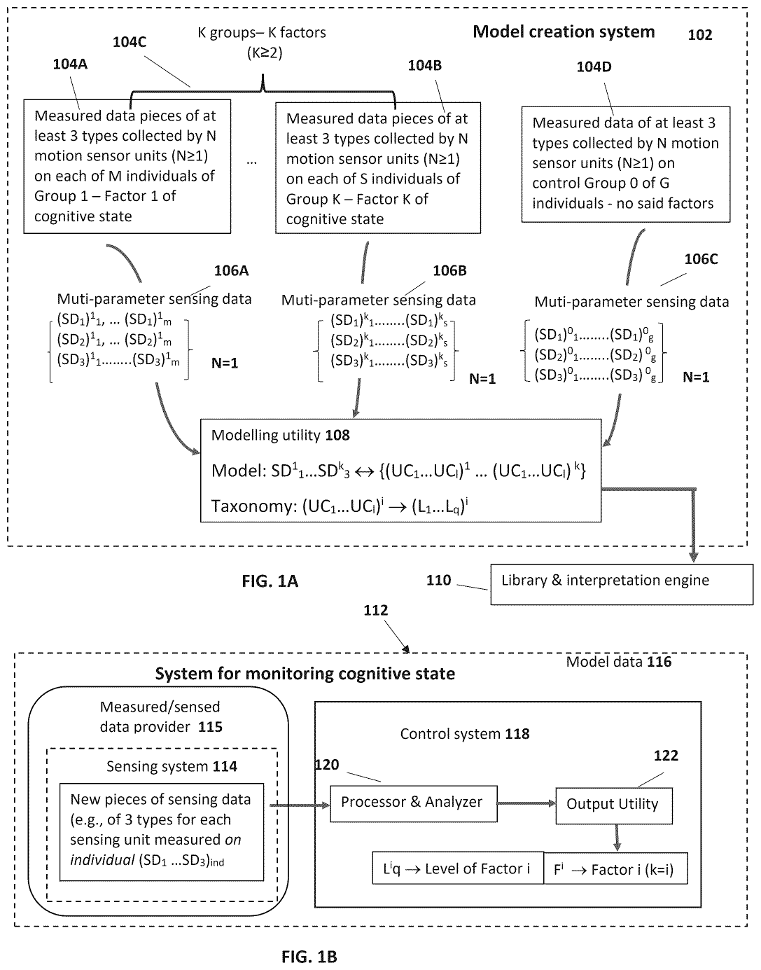

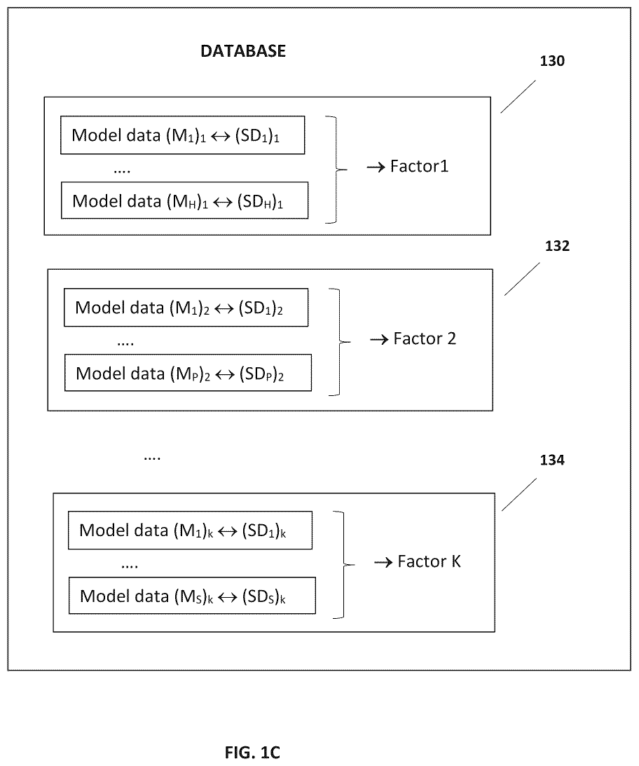

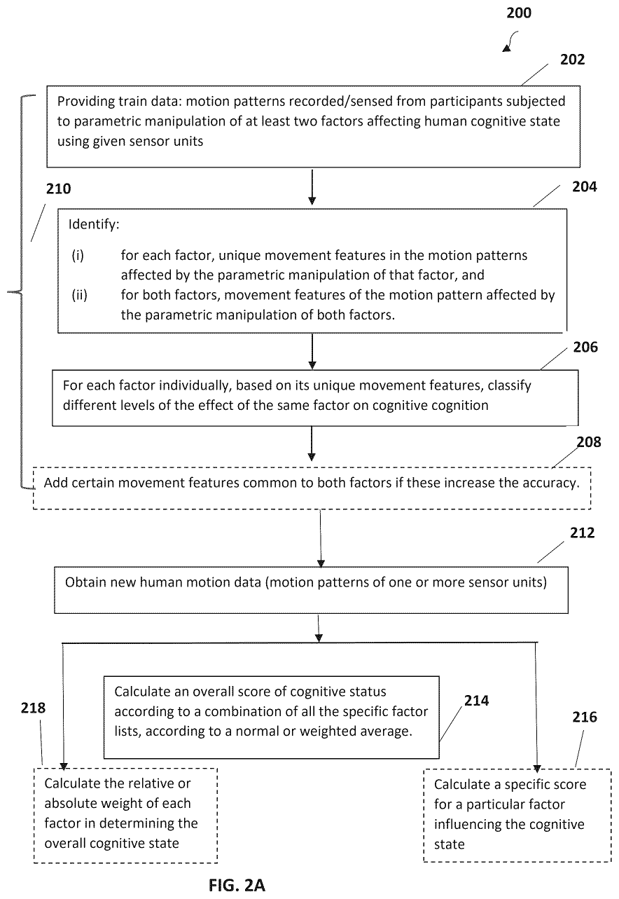

Reference is made to FIGS. 1A and 1B exemplifying, by way of block diagrams, the technique of the present disclosure for identifying a cognitive state of an individual.

A monitoring system 112 of the present disclosure illustrated schematically, by way of a block diagram exemplified in FIG. 1B, utilizes machine learning model(s) previously created by a model creation system 102 whose operation is exemplified in FIG. 1A. The model(s) is/are created once during a learning/training stage, for each individual's activity and for given types of sensing data, i.e. measuring conditions, and is/are properly stored in a database 110. Such database includes a library with its data interpretation engine. The monitoring system 112 can then repeatedly apply such model(s) to newly (real time) measured pieces of data collected under the respective measuring conditions and relating to the corresponding activity of the individual, to monitor a cognitive condition of the individual in order to properly detect/identify a specific cognitive state of the individual. The cognitive state is indicative of a change in the cognitive condition associated with/caused by one or more characteristic factors.

The model creation system 102 uses N (N≥1) sensor units to measure motion patterns originating in at least one body part of an individual. As indicated above, the sensor unit(s) may include a sensor unit of the type directly measuring the motion originated at the body part of the individual (e.g. sensor physically attached to the body part) and/or a sensor unit of the type measuring motion of a device/element interacting with the body part of the individual and being driven by said body part. One typical example of the sensor associated with a device/element driven by the individual is a sensor attached to or integrated in a vehicle's wheel being operated by a vehicle's driver.

Specifically, in some embodiments, during the training stage, training data set is generated by performing measurements on K groups of individuals (K≥2) under certain activity, where each group includes multiple individuals whose cognitive state is characterized by at least one of K different factors. Also utilized in the training stage is a so-called “control group” or “Group 0” of multiple individuals whose cognitive state is free of any of K factors, e.g. whose cognitive condition is normal with respect to a certain activity.

In some other embodiments, during the training stage, a separate algorithm is trained on different levels of each cognitive state (e.g., algorithm for different levels of alcohol and algorithm for different levels of fatigue). In this case, the control group(s) might not be used (i.e., group with zero amount of the factor of interest). For example, a blood alcohol level (BAC) of 0.05 can serve as a control group for BAC level of 0.08.

In the nonlimiting example of FIG. 1A, the measured data (indicative of/corresponding to the sensing data) is collected from M individuals of a first group and corresponds to a cognitive state characterized by Factor 1 (e.g. drunkenness), and so on up to the K-th group of S individuals corresponding to a cognitive state characterized by Factor K (steps 104A, 104B). For each individual in each group, measured data pieces (motion patterns collected over time) are collected by each of said N motion sensors.

The measured data pieces include at least two types of data relating to, respectively, at least two different measuring conditions. These may for example include at least two of the following: different sampling rates, different resolution (accuracy) of measurements, and different (angular/linear) dimensions. In this specific not limiting example, three types of sensing data SD₁, SD₂, SD₃ corresponding to these three different measuring conditions are collected.

Thus, in the case of a single sensor (N=1), the measured data relating to the M individuals of the 1^{st} group, characterized by a specific factor of cognitive state (k=1) includes the sensing data formed by three types of measured data pieces 106A:
(SD₁)¹₁, . . . (SD₁)¹ₘ
(SD₂)¹₁, . . . (SD₂)¹ₘ
(SD₃)¹₁ . . . (SD₃)¹ₘ
   or generally {(SD₁)¹₁, (SD₂)¹₁, (SD₃)¹₁}. This multi-parameter sensing data 106A is transferred to a modelling utility 108.

In a similar way, measured data pieces 106B are collected by the sensor unit on the K-th group of S individuals, characterized by factor K (e.g. exhaustion) of cognitive state which includes:
(SD₁)^{k}₁ . . . (SD₁)^{k}ₛ
(SD₂)^{k}₁ . . . (SD₂)^{k}ₛ
(SD₃)^{k}₁ . . . (SD₃)^{k}ₛ
   or generally {(SD₁)^{k}₁, (SD₂)^{k}₁, (SD₃)^{k}₁} and this multi-parameter sensing data (106B) is transferred to the modelling utility 108.

In total, K groups of individuals are selected for the training stage 104C, corresponding to K characteristic factors, each factor representing a specific cognitive state of an individual.

As indicated above, in some embodiments, the train data may also include measured data collected by N (N≥1) sensor units on the control group (Group 0) of G individuals whose measured data 104D corresponds to the cognitive state not characterized by any of the factors 1 . . . . K and the respective multi-parameter sensing data 106C are transferred to the modelling utility 108. This sensing data includes:
(SD₁)⁰₁ . . . (SD₁)⁰₈
(SD₂)⁰₁ . . . (SD₂)⁰_{g}
(SD₃)⁰₁ . . . (SD₃)⁰_{g}
   or generally {(SD₁)⁰₁, (SD₂)⁰₁, (SD₃)⁰₁}.

It should be understood that all the above-mentioned measured data pieces are indicative of motion patterns collected over time.

In some embodiments, the model creation system 102 is configured and operable to analyze, at the model learning/training stage, the measured data obtained by sensor units of various types (i.e. different sensing types and/or the same sensing type but associated with different body parts of individuals) while at the “normal” cognitive condition of individuals at the certain activity of the individuals. Then, the models are trained to identify a change in such measured/sensing data affected by a change in the cognitive condition from the normal one, and the so-obtained sensing data can be classified between different levels of the cognitive condition change. This may be further used by the monitoring system to selectively apply/initiate the model-based processing of “real time” measured data upon detecting certain level of the change in the cognitive condition of the individual, using the predetermined model, to identify a cognitive state of the individual (i.e. factor being a source of the change of the cognitive condition), and preferably also determine the level of the respective factor.

The modeling utility 108 is configured and operable to apply machine learning processing to the sensing/measured data 106A and 106B (and possibly also 106C) and generate at least one model interpreting said sensing data in association with at least one predetermined characteristic factor corresponding to a cognitive state of an individual. The modelling utility 108 determines, for each tested i-th factor from said K factors, a set of L characteristics (UC₁ . . . . UCₗ)ⁱ uniquely describing a change in a cognitive condition of an individual, caused by a certain cognitive state of said individual associated with said i-th characteristic factor.

In addition, for each tested i-th characteristic factor individually, based on its associated unique movement features, a taxonomy (i.e. classification) (L₁ . . . . L_{q})ⁱ of the different levels of the effect of the same factor on cognitive cognition is preferably created. The output of the modelling utility, including the model(s) and preferably also the taxonomy, is/are saved in a library which is typically associated with an interpretation engine 110 for future use by the monitoring system 112 during “online” operational mode for monitoring a cognitive state of “real” individual.

As shown in FIG. 1B, the monitoring system 112 of the present disclosure includes a control system 118 which is configured (i.e., includes a data communication port/utility) to communicate with measured/sensed data provider 115 and to communicate with database 110. The measured data provider may be a storage device where the sensed data is stored; such storage device 115 may be memory of a separate system associated with a sensing system 114, or the internal memory of the sensing system 114. In some embodiments, the monitoring system 112 may include the sensing system 114.

The sensing system 114 includes at least one sensor unit which is configured to measure motion patterns. As described above, the sensing system is configured and operable to provide measured data indicative of motion patterns detected over time from at least one body part of an individual, being measured directly from said body part or a device being operated in association with said body part.

For example, the sensing system operates to measure motions originated at an individual while driving a vehicle. In this example, the movement (motion patterns) being detected may include one or more of the following: the angle of a steering wheel; image data from a camera that photographs the body; measured data provided by a radar that transmits signals to the body to detect movements in the vehicle.

Generally, there may be multiple number N of sensor units, which may include sensor units of the same type associated with different body parts of the individual and/or may include sensors of different types associated with the same body part of the individual, as the case may be. Thus, generally, the sensing data may include multiple data portions/pieces relating to a plurality of N sensor units, (SD₁ . . . . SD_{N})_{ind}, where each such sensing data piece includes the above-described at least two or preferably three measured data pieces relating to the respective number of different measuring conditions.

However, the inventors have shown that using a single sensor unit collecting motion patterns originating in a respective body part of an individual, while under at least two (preferably three) types of measuring/sensing conditions, can be sufficient to provide meaningful measured data enabling identification of the cognitive state of the individual.

The control system 118 is configured as a computerized system, receiving as an input the measured data pieces from the measured data provider 115 (e.g., the sensing system 114) and including inter alia data processor and analyzer 120, and output utility 122.

As noted above, the control system 118 may be integral with the sensing system 114 or may be a separate system configured for data/signal communication with the sensing system 114 or generally measured data provider where sensing data, generated by the sensing system 114, is stored. To this end, any known suitable type of data communication can be used, which is known per se and therefore may not be specifically described.

The control system 118 is also configured to access data stored in the database 110. This data may be stored in a memory of the control system or in a separate storage device to which the control system has access via any known suitable communication technique.

It should also be noted that, depending on the individual's activity being monitored and/or a cognitive state to be identified, the control system may also be configured and operable to record data indicative of the monitoring results and properly transfer such data to and/or allow access to such data for an authorized person/entity. In addition, information can be used to implement an intervention that is specifically relevant to the factor that caused the cognitive decline. For example, in case of fatigue shake the chair; in case of drunkenness slow down the speed of travel.

The processor and analyzer 120 of the control system 118 receives the measured data from the sensing system 114 and utilizes a predetermined model data 116 stored in the library with its associated interpretation engine 110. As described above, the model describes a relation between motion patterns, collected by the sensor unit of a given type under predetermined measuring condition(s) and in relation to a given body part type, from an individual, and multiple characteristics/features of motion patterns associated with/assigned to at least one predetermined characteristic factor corresponding to the cognitive state of an individual. By this, the cognitive state of the individual being monitored can be identified.

Reference is made to FIG. 1C exemplifying the database/library structure. It should be noted that the library 110 may contain model data based on variable numbers of sensor units/measuring conditions per characteristic factor, providing the respective variable number of sensing data (SD). For example, the model data 130 assigned to characteristic Factor 1 may be based on sensing data obtained from H sensor units (operable under given measuring conditions), whereas model data 132 assigned to characteristic Factor 2 may be based on sensing data obtained from P sensor units, where HP. Respectively, model data 134 assigned to the characteristic Factor K may be based on sensing data obtained from S sensor units, where H≠P≠S.

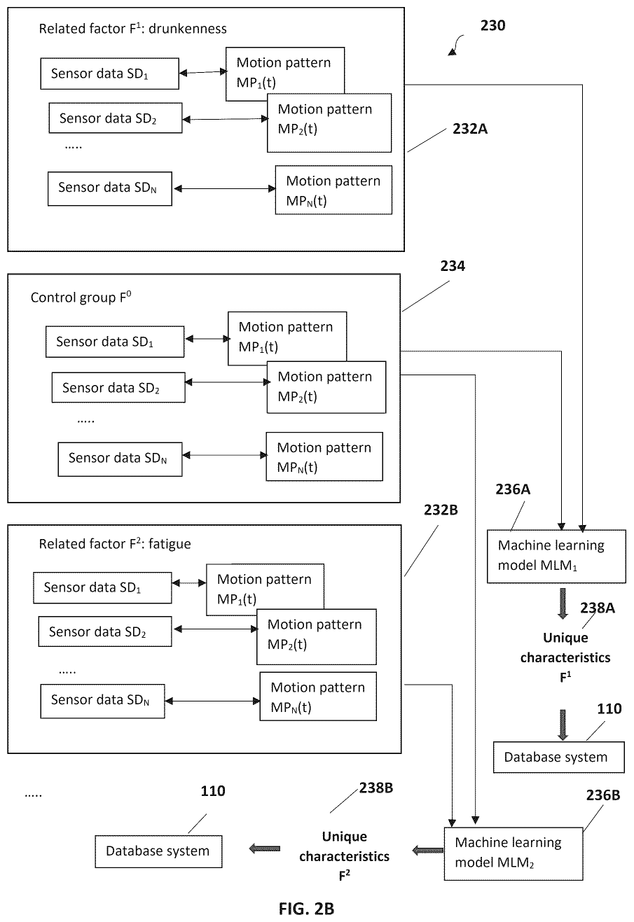

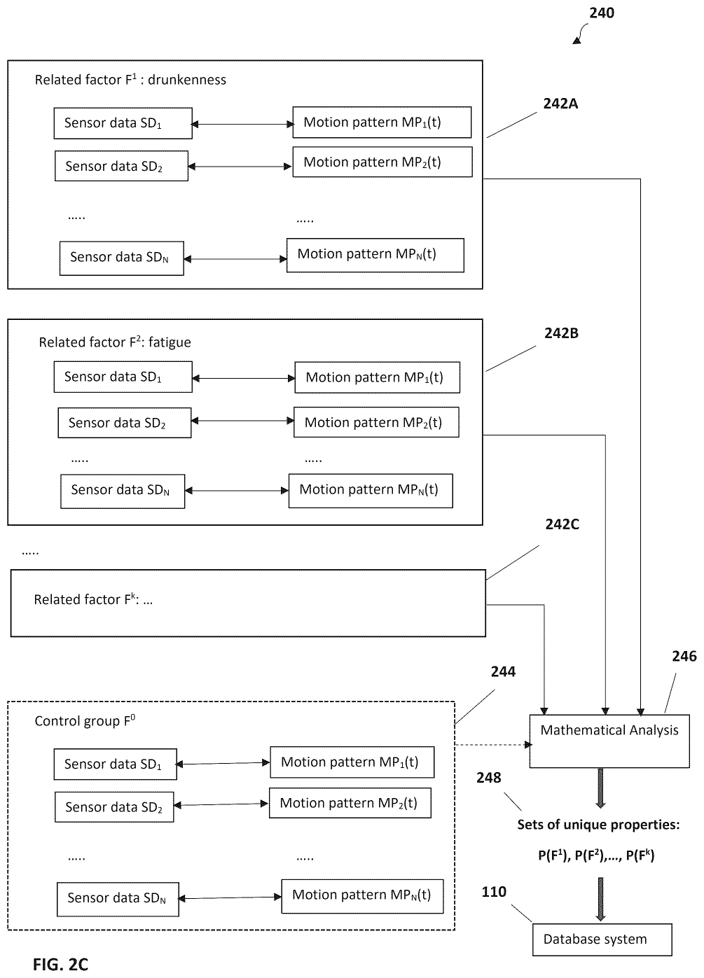

Reference is made to FIG. 2A exemplifying by way of a flow diagram 200 a method of the present disclosure to identify the cognitive state (the cause of alteration of the cognitive condition from normal one) and enhance the algorithm ability to quantify the change of the cognitive condition. It should be noted that the description of this example actually includes many possible options for the implementation of the data processing algorithm. Therefore, the exemplified order of the method steps described herein should not be limiting (the described order of events is not mandatory).

A learning mode is conducted (step 202), involving parametric manipulation of at least two factors affecting human cognitive state while recording motion patterns collected from the participants using the proper sensor units. The recorded motion patterns are analyzed (step 204), as will be described in more detail below, to find unique movement features affected by the parametric manipulation of that characteristic factor and movement features affected by the parametric manipulation of both characteristic factors. For each characteristic factor individually, based on the respective unique movement features, a taxonomy of different levels of the effect of the same characteristic factor on cognition is created (step 206). Certain unique movement features that reduce accuracy can be omitted. If certain movement features, common for both characteristic factors, are found to increase the accuracy, they can be added (step 208). The learning mode, its analysis and the creation of taxonomy, is considered as a preparatory stage (step 210) and is performed only once with the chosen common (cognitive) characteristic factors, e.g., drunkenness, fatigue, etc.

Thus, when running the algorithm (model-based processing of the measured data), it collects indications for relevant motion patterns from the specific characteristic factors' lists it has and from the non-specific factor lists. A new human motion data is obtained (step 212).

The algorithm can give an overall score of cognitive status according to a combination of all the specific characteristic factors' lists, according to a normal or weighted average (step 214). Alternatively, the algorithm can give a specific score for a particular characteristic factor influencing the cognitive state (step 216) or provide the relative or absolute weight of each characteristic factor in determining the overall cognitive state (step 218).

Reference is made to FIGS. 2B and 2C describing two non-limiting examples of a learning stage according to the present disclosure to create a taxonomy of different levels of the effect of the same characteristic factor on cognition.

In the first example, described by a flow diagram 230 in FIG. 2B, motion patterns, MPᵢ(t), embedding motor information (e.g. about goal directed movements) of a person are gathered by at least one sensor unit providing sensing data SD₁ for at least two groups of individuals. Here, each group is characterized by a common characteristic factor Fⁱ (e.g., F¹=drunkenness (stage 232A)) or common characteristic F²=fatigue (stage 232B)), that characterizes the cognitive state of the individuals of said group and does not characterize the other group.

It should be understood that sensing data SD₁ contains data pieces of certain types (e.g., at least 3 types corresponding to different measuring/sensing conditions, as described above.

In total, K groups of individuals representing K characteristic factors (K≥2) are measured as described above. Data gathering is done while an individual is performing a particular task (activity) and specific motor information (motion patterns) describing movements (e.g., goal directed) that help to perform the task is gathered. In a specific not-limiting example, while driving a vehicle, this information may include movements that move the steering wheel, or motion reactions to road conditions or vehicle movement, but not falling of the head or closing eyelids. In all groups, the data MPᵢ(t) is of the same type of sensor unit (e.g., in all groups steering wheel movements are collected while driving).

One or more control groups, denoted F⁰, may be added (step 234), each having no characteristic change in cognitive or emotional state. As indicated above, the provision of the control group(s) is optional.

It should be noted that each sensor data (SD) in each group, relating to a common characteristic factor Fⁱ of cognitive state (steps 232A, 232B, 234), is gathered from multiple individuals for the purpose of strengthening the statistical confidence of data analysis (as described and shown by elements 106A, 106B, 106C in FIG. 1A). This is not illustrated in FIG. 2B for clarity of presentation.

The motion patterns, MPᵢ(t), of each i-th experimental groups having characteristic factor Fⁱ, may be separately classified utilizing data about the motion patterns MPᵢ(t) of the control group F⁰. As mentioned above, it is possible to perform classification based on so-called “reference data” of more than one control groups, each of which indicates a different level (amount or intensity) of the respective factor.

Then, machine learning or similar methods are used for classification between the groups to distinguish between the groups (e.g. distinguish between each experimental group associated with a specific single characteristic factor and the control group), thus creating respective models (e.g., machine learning models) MLM₁(step 236A) and MLM₂ (step 236B), corresponding to the two different common factor groups. The machine learning model identifies unique characteristics (steps 238A and 238B) in the comparison of each of the experimental groups (related to the specific characteristic factor being monitored) versus the respective control group. It is also possible to implement the algorithm in order do distinct between groups for single characteristic factor (i.e., fatigue) or in order to distinguish between groups of multiply characteristic factors (i.e., fatigue and drunkenness). The unique characteristics, in association with the respective characteristic factor, are stored in the database system 110 for future use during monitoring of individual's cognitive state as described above.

In the flow diagram 240 of the second example shown in FIG. 2C, similar to the first example of FIG. 2B, motion patterns, MPᵢ(t), indicative of movements originated in a body part of an individual are gathered by at least one sensor unit providing sensor data (SD) including measured data pieces corresponding to different measuring conditions, for each individual from at least two groups of individuals, where each group is characterized by a common characteristic factor Fⁱ(e.g., F^{l}=drunkenness (step 242A) or F²=fatigue (step 242B)), that corresponds to the cognitive state of the individuals of the respective group and does not characterize the cognitive state of the individuals of the other group.

In total, K groups of individuals representing K characteristic factors (K≥2) are measured. However, in this example of FIG. 2C, mathematical analysis is performed (step 246) to identify unique sets of properties, P(F¹), P(F²), . . . , P(F^{k}), that are unique for the motion patterns measured on individuals of one group and are not found in measured data of another group (248). A control group, F⁰, that is not characterized by any of these characteristic factors that affect/change the individual's cognitive status can be added to the comparison (244). The sets of unique properties are stored in the database system 110 for future use during monitoring of individual's cognitive state as described above.

It should be noted that each sensor data (SD) in each group, characterized by a common characteristic factor Fⁱ of cognitive condition (steps 242A, 242B, 242C, 244), is gathered from multiple individuals for the purpose of strengthening the statistical confidence of data analysis (elements 106A, 106B, 106C in FIG. 1A), but this is not illustrated in FIG. 2C for clarity of presentation.

In addition, the unique features discovered in the comparisons described above can be used to improve the ability to identify cognitive change in general. This is because motor characteristics that are not unique to the characteristic factor of interest (e.g. fatigue or intoxication) may affect both the algorithm's training phase and its real-time work phase and contaminate both the learning phase and real-time reading.

For example, the experiments conducted by the inventors have shown that fatigue often intensifies as a person consumes more alcohol. If so, motor characteristics indicative of a high level of fatigue may appear during experimental parametric manipulation of high levels of intoxication. As a result, the algorithm that is supposed to detect drunkenness might actually detect an additive effect of fatigue and drunkenness on the driver's movement. As a result, both the ability to train the algorithm to accurately detect the level of alcohol in the driver's blood and the ability to accurately detect in real time the level of alcohol in the driver's blood might be compromised.

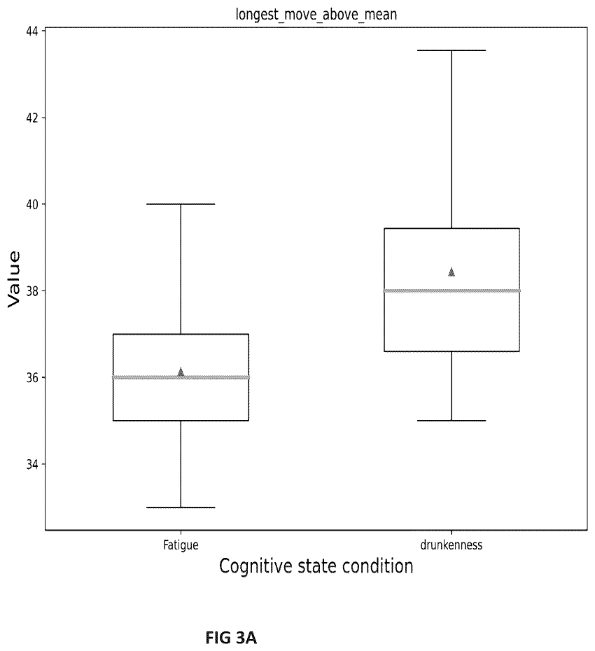

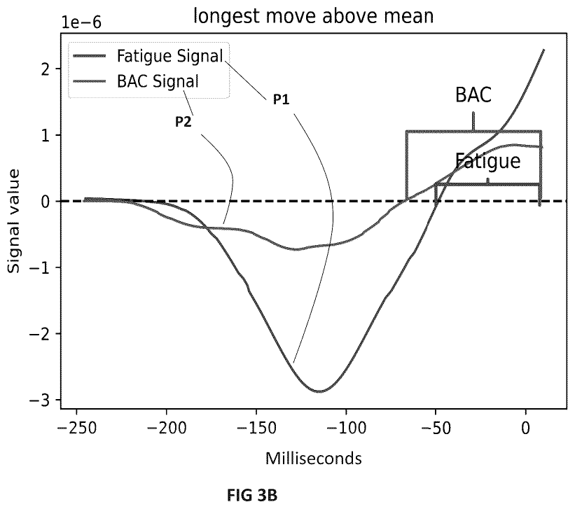

Reference is made to FIGS. 3A and 3B exemplifying the values of the “longest move above mean” feature extracted from individual's motion patterns/signals, i.e., the length of the longest sub-signal that is higher than the average value of the signal. FIG. 3A shows the distribution of the value (i.e., “Longest move above mean”) for the different cognitive states (fatigue and drunkenness), and FIG. 3B shows an example of two signals P₁ and P₂ being, respectively, fatigue and blood alcohol content (BAC) signals, with different values for the “Longest move above mean” (the average of both signals is close to zero). The BAC signal P₂ measured on an individual who drank more than 3 doses of alcohol (blood alcohol content, BAC, is 0.08% or higher) is longer (i.e., only the signal part above zero is considered) than the value measured on an individual under extreme fatigue condition-signal P₁.

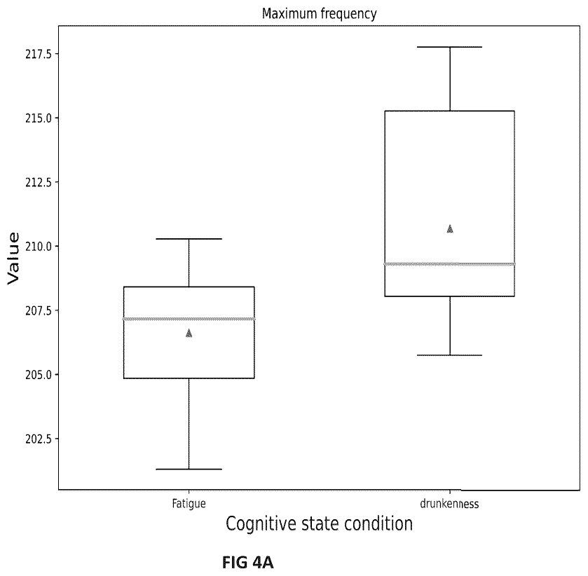

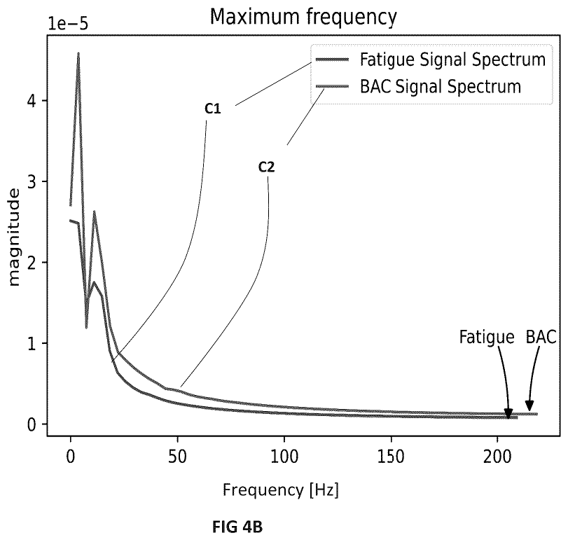

Reference is made to FIGS. 4A and 4B exemplifying the feature of “Maximum frequency” of measured signal expressed by the 95th percentile of the signal frequency. FIG. 4A shows the distribution of the value corresponding to the 95th percentile of the signal frequency for two different cognitive states (fatigue and drunkenness), and FIG. 4B shows the frequency spectra of two signals C₁ and C₂ belonging to two individuals with different cognitive states and, respectively, having different values for the “Maximum frequency”. The “Maximum frequency” of the signal (designated by arrows in FIG. 4B) of an individual who drank more than three doses of alcohol (blood alcohol content, BAC, is 0.08% or higher) is bigger than the respective value of an individual under extreme fatigue condition.

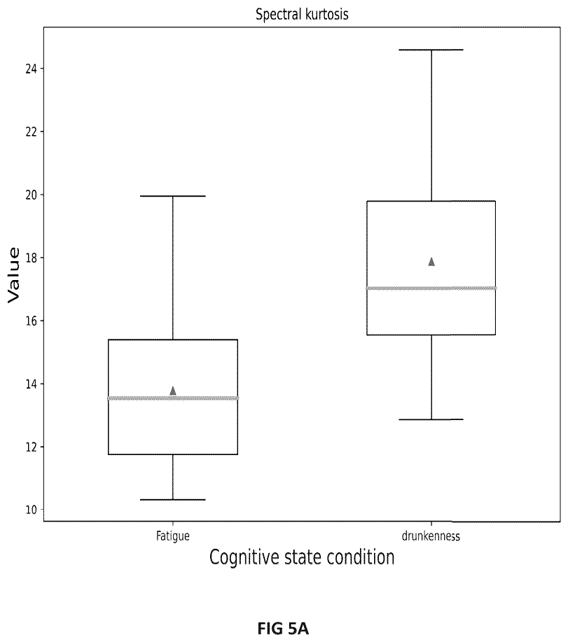

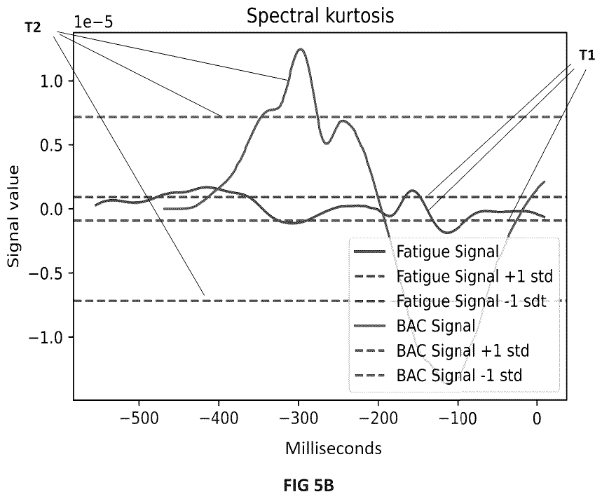

Reference is made to FIGS. 5A and 5B exemplifying the parameter/feature of spectral kurtosis which measures the “tailedness” of a signal's frequency distribution. The spectral kurtosis (SK) is a statistical tool which can indicate the presence of series of transients and their location in the frequency domain. Originally, SK was defined as the normalized fourth-order moment of the magnitude of the short-time Fourier transform. Methods based on the power spectrum of the frequency distribution may fail in detecting outlier contribution to certain frequencies due to averaging operations involved in power spectral density calculation. SK can be calculated as the normalized fourth-order moment of the signal's frequency distribution.

FIG. 5A shows the distribution of the value (i.e., SK/“tailedness” of signal's frequency distribution) for the different cognitive states (fatigue and drunkenness), whereas FIG. 5B shows two signals R₁ (and its “+” and “−” statistic deviations) and R₂ (and its “+” and “−” statistic deviations) with different frequency kurtosis measured on, respectively, an individual under extreme fatigue condition, and an individual who drank more than three doses of alcohol (blood alcohol content, BAC, is 0.08% or higher). As shown in the figure, the signal R₁ measured on the individual under extreme fatigue condition is more concentrated compared to the signal R₂ sampled from a motion pattern measured on the individual who drank more than three doses of alcohol (blood alcohol content, BAC, is 0.08% or higher).

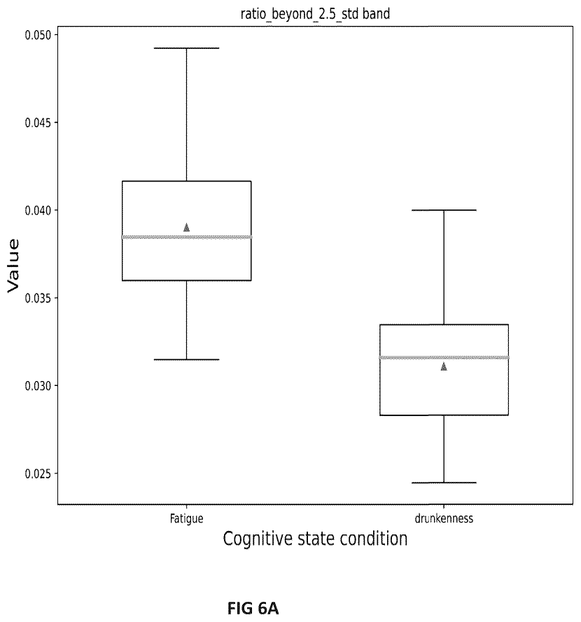

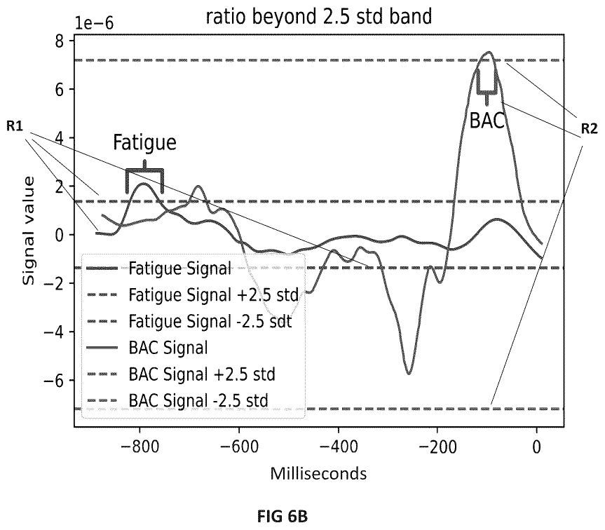

Another statistical characteristic which was found by the inventors to distinguish between two specific factors (drunkenness and fatigue) known to change cognitive or emotional state, is shown in FIGS. 6A and 6B. The inventors analyzed the ratio of values in the signal (motion pattern) which are higher than 2.5 times the standard deviation (std) of the signal. FIG. 6A shows the distribution of the value (“ratio beyond 2.5 std”) for the different cognitive states, having fatigue and BAC characteristic factors. FIG. 6B shows two signals R₁ and R₂ (and their “+” and “−” statistic deviations) with different “ratio beyond 2.5 std”: the signal R₁ measured on an individual under extreme fatigue condition has higher ratio than the ratio in the signal R₂ which sampled from movement of an individual who drank more than three doses of alcohol (blood alcohol content, BAC, is 0.08% or higher).

It should be noted that the unique features exemplified in the comparisons detailed above are not limiting. Some additional features may be used to distinguish between specific factors, These additional features may include one or more of the following: the length of the longest sub signal, i.e. “longest move above mean” (that is lower than the average value of the sub signal or portion/part of the signal); entropy of the signal power for a given frequency range (i.e. a measure of the signal randomness); Number of sign changes of a signal in a given period of time; Pearson's correlation coefficient for the OLS (Ordinary Least-Squares) model; the average of the signal differential values in given range of the signal's distribution.

Table 1 below exemplifies a preliminary demonstration of the accuracy of cognitive state detection using the technique of the present disclosure:

TABLE 1

| | |
|---|---|
| Model Input | Model Output |

| | No. of | 0 cc alcohol/ | | |
|---|---|---|---|---|
| Factor | Samples | Not tired | Tired | Intoxicated |
| 0 cc alcohol/ | 147 | 80% | 8% | 12% |
| Not tired | | | | |
| Tired | 39 | 38% | 62% | 0% |
| Intoxicated | 24 | 25% | 0% | 75% |

The sensing data includes measurements of the steering wheel angular velocity with a sampling rate of 500 Hz and resolution (accuracy) of 0.02 degrees. A model was created to distinguish between two characteristic factors characterizing human cognitive states: tiredness and intoxication. Training of the model was performed by recording motion patterns sensed from participants subjected to parametric manipulation of these two factors.

In the next stage, the model was applied to the following test cases:

(1) 147 individuals having zero alcohol (i.e., non-intoxicated) and not tired. Analysis of the measured motion patterns using the trained model resulted in correctly identifying the absence of both factors in 117 individuals out of 147 (80%), while 12 individuals (8%) were identified as tired, and 18 individuals were identified as intoxicated.

(2) 39 individuals known to be tired. Analysis of the measured motion patterns using the trained model resulted in correctly identifying the cognitive state of tiredness in 24 individuals (62%), while 15 (38%) individuals were identified as lacking any of the two factors and zero individuals were identified with intoxication.

(3) 24 individuals known to be intoxicated. Analysis of the measured motion patterns using the trained model resulted in correctly identifying the cognitive state of intoxication in 18 individuals (75%), while 6 (25%) individuals were identified as lacking any of the two factors and zero individuals were identified with tiredness.

These results show a very good specificity of distinguishing between the two factors, tiredness and intoxication.

1. A system for monitoring a cognitive state of an individual during a certain activity of the individual, the system comprising:
a control system configured for data communication with a measured data provider to receive therefrom measured data indicative of motion patterns originated in at least one body part of the individual and being detected over time by a sensing system operable with predetermined measuring conditions and comprising at least one of the following: a sensor unit physically attached to the body part, a sensor unit associated with a device interacting with the body part; and a radar transmitting signals to the body, the control system being configured and operable to process the measured data by applying thereto model-based processing and determine a cognitive state of the individual, said model-based processing comprising applying to said measured data a number of predetermined models, each of the predetermined models describing a relation between motion patterns, originated in at least one body part of an individual and being collected under predetermined measuring conditions, and an associated characteristic factor of a cognitive state being a source affecting a change of a cognitive condition of the individual from a normal condition during certain activity, said characteristic factor describing the cognitive state of the individual, distinguishing from different factors affecting cognitive conditions of individual.

2. The system according to claim 1, wherein said control system comprises a processor and analyzer adapted to be responsive to the measured data and to analyze said measured data by applying thereto at least one selected model from said number of models, in accordance with the cognitive state to be detected, and determine the cognitive state by identifying in the measured data at least one feature uniquely associated with the respective characteristic factor describing a cognitive condition of the individual during certain activity.

3. The system according to claim 21, wherein said control system has one of the following configurations:
comprises processor and analyzer is configured to analyze said measured data and, upon identifying a predetermined level of change in a cognitive condition of the individual being monitored, applying at least one selected model from said number of models to said measured data to identify the predetermined characteristic factor affecting said change of the cognitive condition, and extracting the corresponding cognitive state of said individual; and
comprises processor and analyzer responsive to the measured data corresponding to a predetermined level of change in a cognitive condition of the individual being monitored, to apply at least one selected model from said number of models to said measured data to identify the predetermined characteristic factor affecting said change of the cognitive condition, and extracting the corresponding cognitive state of said individual.

4. The system according to claim 31, wherein said control system comprises processor and analyzer configured and operable to analyze said measured data and, upon identifying a predetermined level of change in a cognitive condition of the individual being monitored, applying said at least one selected model to said measured data to identify the predetermined characteristic factor affecting said change of the cognitive condition, and extracting the corresponding cognitive state of said individual; and quantifying a degree of change of a cognitive condition of the individual associated with said corresponding cognitive state.

5. (canceled)

6. The system according to claim 1, characterized by at least one of the following:
said control system is configured and operable for data communication with a storage device storing said number of predetermined models via a communication network;
said number of models comprises at least one machine learning model obtained in a training and learning procedures.

7. (canceled)

8. The system according to claim 1, characterized by at least one of the following:
said at least one predetermined characteristic factor corresponds to the at least one of the following cognitive states: drunkenness, fatigue, exhaustion, stress, motion sickness, inattention, development of acute physical condition affecting cognitive state;
the characteristic factor characterizing the cognitive state comprises at least one of the following: drug use, inattention, acute physical condition, exhaustion, intoxication, fatigue, stress, attention withdrawal, motion sickness, drunkenness;
the characteristic factor comprises one of fatigue and drunkenness of an individual while driving a vehicle, the measured data being indicative of the motion pattern detected from measurements of steering wheel angular velocity.

9. The system according to claim 1, further comprising the sensing system comprising a number N of sensor units (N≥1), each sensing unit comprising one or more sensors and being configured and operable with predetermined measuring conditions to detect data indicative of the motion patterns originated in at least one body part of the individual and generate said measured data indicative of the motion patterns being detected.

10. (canceled)

11. (canceled)

12. The system according to claim 2, wherein the characteristic factor comprises one of fatigue and drunkenness of an individual while driving a vehicle, the measured data being indicative of the motion pattern detected from measurements of steering wheel angular velocity, said at least one feature uniquely associated with the characteristic factor comprising at least one of the following: longest move above mean determined as a length of the longest sub-signal in the motion pattern that is higher than an average value of a measured motion pattern signal; maximum frequency of a measured motion pattern signal; tailedness of frequency distribution of a motion pattern signal being measured; and a ratio of values in the motion pattern signal which are higher than 2.5 times standard deviation of the signal.

13. A method for monitoring a cognitive state of an individual during a certain activity of the individual, the method comprising:
providing measured data indicative of motion patterns originated in at least one body part of the individual and being detected over time under predetermined measuring conditions by at least one sensor unit comprising at least one of the following: a sensor unit physically attached to the body part or a device interacting with the body part; and a radar transmitting signals to the body,
processing the measured data by applying thereto model-based processing and determining a cognitive state of the individual, said model-based processing comprising applying to said measured data a number of predetermined models, each of the predetermined models describing a relation between motion patterns, originated in at least one body part of an individual and being collected under predetermined measuring conditions, and an associated characteristic factor being a source affecting a change of a cognitive condition of the individual from a normal condition during certain activity, said characteristic factor describing the cognitive state of the individual, distinguishing from different factors affecting cognitive conditions of individual.

14. The method according to claim 13, wherein said processing comprises identifying in the measured data at least one feature uniquely associated with the respective characteristic factor describing a change in cognitive condition of the individual during certain activity.

15. The method according to claim 14, wherein said processing comprises analyzing said measured data and, upon identifying a predetermined level of change in the cognitive condition of the individual being monitored, applying said at least one selected model to said measured data to identify the predetermined characteristic factor affecting said change of the cognitive condition, and extracting the corresponding cognitive state of said individual.

16. The method according to claim 15, wherein said processing comprises quantifying a degree of change of the cognitive condition of the individual associated with said corresponding cognitive state.

17. The method according to claim 13, wherein said providing of the measured data comprises one of the following:
communication, via a communication network, with a storage device storing said number of predetermined models; and performing measurements by one or more sensors under predetermined measuring conditions to detect data indicative of motion patterns originated in at least one body part of the individual, and
generating said measured data indicative of the motion patterns being detected.

18. (canceled)

19. The method according to claim 13, characterized by at least one of the following:
said predetermined number of models comprises at least one machine learning model obtained in a training and learning procedures;
said at least one predetermined characteristic factor corresponds to the at least one of the following cognitive states: drunkenness, fatigue, exhaustion, stress, motion sickness, inattention, development of acute physical condition affecting cognitive state;
the characteristic factor characterizing the cognitive state comprises at least one of the following: drug use, inattention, acute physical condition, exhaustion, intoxication, fatigue, stress, attention withdrawal, motion sickness, drunkenness;
the characteristic factor comprises one of fatigue and drunkenness of an individual while driving a vehicle, the measured data being indicative of the motion pattern detected from measurements of movements in a vehicle being driven by the individual.

20. (canceled)

21. (canceled)

22. The method according to claim 14, wherein the characteristic factor comprises one of fatigue and drunkenness of an individual while driving a vehicle, the measured data being indicative of the motion pattern detected from measurements of steering wheel angular velocity of a vehicle driven by the individual, said at least one feature uniquely associated with the characteristic factor comprises at least one of the following: longest move above mean determined as a length of the longest sub-signal in the motion pattern that is higher than an average value of a measured motion pattern signal; maximum frequency of a measured motion pattern signal; tailedness of frequency distribution of a motion pattern signal being measured; and a ratio of values in the motion pattern signal which are higher than 2.5 times standard deviation of the signal.

23. (canceled)

24. A system for use in monitoring a cognitive state of an individual during a certain activity of the individual, the system being a computer system comprising:
modeling utility configured and operable to apply machine learning processing to sensing data and generate at least one model interpreting said sensing data in association with at least one predetermined characteristic factor corresponding to a cognitive state of an individual, wherein:
said sensing data corresponds to measurements performed by N (N≥1) sensing units in association with body parts of individuals comprising at least one of the following: at least one sensing unit physically attached to the body part; at least one sensing unit associated with a device interacting with the body part; and a radar transmitting signals to the body; each measurement being indicative of motion patterns originated at a respective body part of an individual, said sensing data comprising a train data formed by at least first and second sets of motion patterns measured on, respectively, at least one first group of a plurality of M individuals whose cognitive state is characterized by said at least one predetermined characteristic factor, and at least one second group of a plurality of G individuals whose cognitive state is free of said at least one predetermined characteristic factor, the characteristic factor being a source affecting a change of a cognitive condition of the individual from a normal condition during certain activity of the individual and distinguishing from different factors affecting cognitive conditions of individual;
said modelling utility is configured and operable to receive said train data and apply model learning and training processing thereto and determine, for each of said at least one predetermined characteristic factor, a set of L characteristics (UC₁ . . . UC_{L}) uniquely describing a change in a cognitive condition of an individual corresponding to a certain cognitive state of said individual.

25. The system according to claim 24, wherein said at least one predetermined characteristic factor corresponds to the at least one of the following cognitive states: drunkenness, fatigue, exhaustion, and stress, motion sickness, inattention, development of acute physical condition affecting cognitive state.

26. The system according to claim 24, wherein characterized by at least one of the following:
said first and second sets of motion patterns correspond to measurements performed on the at least one first group of the individuals whose cognitive state is characterized by said at least one predetermined characteristic factor, and the at least one second group of the individuals whose cognitive state is characterized by a second abnormality factor to be monitored and distinguished from said at least one characteristic factor;
said at least first and second sets of motion patterns correspond to measurements performed on the at least one first group of the individuals whose cognitive state is characterized by said at least one predetermined characteristic factor, and the at least one second group of the individuals being a control group whose cognitive state is classified as a normal state free of each of said at least one predetermined characteristic factor.

27. The system according to claim 2624, wherein said model learning and training processing comprises classifying features of said first and second sets of motion patterns and identifying first and second sets of L characteristics (UC₁ . . . UC_{L})₁ and (UC₁ . . . UC_{L})² uniquely distinguishing the motion patterns measured on the individuals of the two groups.

28. The system according to claim 27, wherein the characteristic uniquely distinguishing the motion patterns measured on the individuals of the two groups is determined by one of the following: (i) absence and presence of a certain motion pattern relating feature in the first and second sets, respectively; or (ii) presence of a certain motion pattern relating feature in both the first and second sets but at different levels according to a predefined criteria.

29. (canceled)

30. A system for monitoring a cognitive state of an individual during a certain activity of the individual, the system comprising: a control system configured for data communication with a measured data provider to receive therefrom measured data indicative of motion patterns originated in at least one body part of the individual and being detected over time by a sensing system being operable with predetermined measuring conditions and comprising at least one of the following: a sensor unit physically attached to the body part; a sensor unit associated with a device interacting with the body part; and a radar transmitting signals to the body, the control system being configured and operable to process the measured data and identify, in said data indicative of the motion patterns, at least one feature uniquely associated with a characteristic factor being a source affecting a change of a cognitive condition of the individual from a normal condition during certain activity, said characteristic factor describing the cognitive state of the individual, distinguishing from different factors affecting cognitive conditions of individual.

31. A system for monitoring a cognitive state of an individual during a certain activity of the individual, the system comprising:
a control system configured for data communication with a measured data provider to receive therefrom measured data indicative of motion patterns originated in at least one body part of the individual and being detected over time by a sensing system operable with predetermined measuring conditions, the control system being configured and operable to process the measured data to identify a characteristic factor being a source affecting a change of a cognitive condition of the individual from a normal condition during certain activity of the individual distinguishing from different factors affecting cognitive conditions of individual, the processing of the measured data comprising:
applying to said measured data a number of predetermined models, each of the predetermined models describing a relation between motion patterns, measured by the sensing system with the predetermined measuring conditions from the body part, and an associated characteristic factor affecting a cognitive condition of the individual; and
generating data indicative of the condition state of the individual during said certain activity of the individual.

32. A system for use in monitoring a cognitive state of an individual during a certain activity of the individual, the system being a computer system comprising:
modeling utility configured and operable to apply machine learning processing to sensing data and generate at least one model interpreting said sensing data in association with at least one predetermined characteristic factor being a source affecting a unique change of a cognitive condition of the individual from a normal condition during certain activity of the individual associated with said characteristic factor, distinguishing from different factors affecting cognitive conditions of individual, and defining a cognitive state of an individual, wherein:
said sensing data corresponds to measurements performed by N (N≥1) sensing units in association with body parts of individuals, being operable with predetermined measuring conditions and comprising at least one of the following: a sensor unit physically attached to the body part; a sensor unit associated with a device interacting with the body part; and a radar transmitting signals to the body, each measurement being indicative of motion patterns originated at a respective body part of an individual, said sensing data comprising a train data formed by at least first and second sets of motion patterns measured on, respectively, at least one first group of a plurality of M individuals whose cognitive state is characterized by said at least one predetermined characteristic factor, and at least one second group of a plurality of G individuals whose cognitive state is free of said at least one predetermined characteristic factor;
said modelling utility is configured and operable to receive said train data and apply model learning and training processing thereto and determine, for each of said at least one predetermined characteristic factor, a set of L characteristics (UC₁ . . . UC_{L}) uniquely describing a change in a cognitive condition of an individual corresponding to a certain cognitive state of said individual.
